(19) United States
(12) Patent Application Publication
(10) Pub. No.: US 2011/0179504 A1
(43) Pub. Date: Jul. 21, 2011

(54) Method For Coral Tissue Cultivation And Propagation

(75) Inventors: Esther Kramarsky-Winter, Rehovot (IL); Yossi Loya, Raanana (IL); Maya Vizel, Kiriat Ono (IL); Craig A. Downs, Amherst, VA (US)

(73) Assignee: Ramot at Tel-Aviv Univeristy Ltd., Tel Aviv (IL)

(21) Appl. No.: 12/678,379

(22) PCT Filed: Sep. 17, 2008
(86) PCT No.: PCT/IL2008/001236

§ 371 Date: Aug. 26, 2010

Related U.S. Application Data
(60) Provisional application No. 60/973,061, filed on Sep. 17, 2007.

(51) Int. Cl. A01K 67/00; C12N 5/02; C12Q 1/02

(52) U.S. Cl. 800/8; 435/383; 435/29

(57) ABSTRACT

This invention relates to a novel method of culturing coral tissues and polyps in vitro. Coral tissues obtained by the method of the invention may be maintained as heterotypic spheroid tissue balls for a period of at least three months or they may be induced to undergo development into new polyps, a process termed re-morphogenesis. This method can produce genetic clones of model species from single individuals that can be propagated either as undifferentiated tissue calli or as developed polyps. The products of the invention are of value to a number of educational, scientific, and commercial endeavors. Specifically, this method can be used to propagate genetic clones (strains) of a model organism for scientific research, to serve as ‘pro-environmental conservation’ sources of coral stock for educational specimens as well as a rapidly generated inventory for commercial aquarium industry. The method of the invention can produce sustainable test lines of corals that could be used to generate risk assessments for the impact of chemicals/activities on coral reefs, as well as being used as part of a regulatory protocol for testing waste effluent and other discharges.

### FIELD OF THE INVENTION

This invention relates to a method of culturing coral explants and polyps in culture.

### BACKGROUND OF THE INVENTION

Scleractinian corals are an ancient group of organisms belonging to the phylum Cnidaria, an evolutionarily basic phylum with tissue grade differentiation (Hyman 1940). The diverse group of Cnidaria includes corals, sea anemones, hydras, jellyfishes, and their relatives. About 9,000 living species are known. The Cnidaria are the simplest Metazoa, and do not even possess organs. All they have is a gastrovascular cavity (digestive and circulatory cavity) and a mouth surrounded by tentacles.

In addition to their recreational and esthetic value, corals are one of the most important components of the world's oceans, providing diverse functions including acting as a sink for atmospheric CO₂, physical protection of shorelines, a habitat for a large number of marine organisms, and a source for potential biological products. Their esthetic and natural value has led a number of national and international companies to become involved in coral culture or farming. For the most part corals are limited to the shallow warm water tropic environments, though recent studies have shown that there are corals in temperate environments in caves and in deep, cold-water environments. Most of the corals collected or farmed to date around the world supply the marine aquarium trade. Cultivated corals have been mainly utilized for natural reef restoration and for the aquarium trade. Recently, there has arisen an interest in the use of corals as model organisms for biological or biomedical research, in a variety of fields including natural product chemistry, ecotoxicology, pharmacology, and developmental biology.

The recent increases in “ruining” of these organisms due to increased demand together with global and local anthropogenic driven changes have caused a loss of these organisms both in terms of species and in terms of biomass. This has resulted in greater restrictions on the collection of these organisms from the wild. These restrictions have led to a variety of attempts to raise corals under culture conditions for commercial purposes such as for the aquarium trade as well as for reef restoration purposes (e.g. Shafir et al 2001, 2006, Arvedlund et al 2003, Latypov 2006). The mariculture or farming of corals has the potential to reduce the number of corals being collected from the wild. The case for culturing corals rather than collecting them is one of conservation and sustainability versus economics. Wild collection offers an instantaneous return on investment, whereas fanning requires investment and a period of time before harvest can begin. However, in the long-run, fanning offers the ability to raise large quantities of corals, reduce operating costs, providing a more sustainable future for the industry in general and for conservation and research as well.

For industry and research purposes, there is a critical need to develop coral models and provide the infrastructure to maintain these living stock collections. Providing well-characterized and documented experimental organisms for researchers and students, as well as for industry, will enable rapid advances through the use of modern techniques to investigate many fundamental biological concepts such as physiology, symbiosis, and development, as well as provide model organisms for testing of novel compounds.

Like other cnidarians, corals possess a tissue grade morphology and are comprised of three layers of soft tissue; an outer epidermis, a gastrodermal layer, and between them a mesoglea, which may or may not contain cells. In addition, scleractinian corals possess an aragonite skeleton. Corals may be solitary or colonial and are capable of sexual and asexual reproduction. They are also known for their morphological and reproductive plasticity, as well as for their innate capacity for regeneration. This capacity enables them to develop new individuals or colonies from fragments of colonies or even from remnant tissues (Krupp et al 1992, Jokiel et al 1993, Kramarsky-Winter and Loya 1996).

Indeed, propagation by fragmentation is a common asexual reproductive mode that is part of the life history trait of many reef-building corals (Highsmith, 1982). Understanding the mechanism behind asexual reproduction holds the key to new and better ways of coral culture. This trait has been manipulated by a number of researchers and aquarists (e.g. Arvedlund et al. 2003, Rinkevich 1995, Bomeman and Lowrie 2001, Shafir et al 2001 and Latypov 2006) which used coral fragments (including tissue and skeleton) to artificially produce large quantities of fragments with high survival rates. These corals have been raised in in situ or ex situ coral nurseries (e.g. Shafir et al. 2001, Borneman 2000, www.drmaccorals.com).

Other studies tried to maintain detached soft tissues in culture. In studies on Pocilloporids, Domart-Coulon et al. (2004) probed the viability of detached soft tissue isolates. Cell viability dropped from 70% to 30% within the first week of maintenance in vitro and no functional polyps were regenerated. Accordingly, short-term isolate cultures limited to 3 days were used in their study

U.S. Pat. No. 6,664,106 discloses a method of culturing cells of sponges and soft corals in vitro. According to this method, aggregates (primmorphs) are formed in culture from a suspension of individual cells. U.S. Pat. No. 6,664,106 does not demonstrate development of the primmorphs into mature corals in culture.

Current methods to cultivate corals are known to have serious limitations including:

(1) Dependence on the use of relatively large space and relatively calm waters for in situ (in the ocean) propagation,

(2) Dependence on large constructions with proper lighting and heating for housing the raceways for ex situ propagation, and

(3) Production of developed colonies that are difficult to maintain in laboratory conditions for long periods of time.

### SUMMARY OF THE INVENTION

The present invention is based on the novel finding that soft tissue fragments devoid of skeleton, obtained from scleractinian corals, can be cultured in seawater-based medium or solution under a relatively low temperature resulting in the formation of spheroids which may be kept viable in the culture for a period of at least several months and may be induced to form a developed coral polyp upon increasing the temperature.

The present invention thus concerns a novel method for tissue culturing of scleractinian corals that may either be maintained as tissue spheroids in culture or be induced to undergo re-morphogenesis into a functional polyp that can undergo asexual propagation and maturation.

According to the invention, tissue fragments are excised from single polyps or from colony fragments (i.e. obtained from single genetic stocks) and are propagated in culture, thus generating coral lines of a single genetic source which develop to mature tissues and/or polyps.

Accordingly by a first of its aspects, the present invention provides a method for obtaining a single genetic source coral tissue culture comprising:
(a) Excision of coral tissue fragments;
(b) Culturing of said tissue fragments in seawater based solution under temperatures that are at the low range for that species/ecotype whereby viable tissue spheroids are obtained; and optionally
(c) Inducing re-morphogenesis of said tissue spheroids into polyps by increasing the culture temperature to a temperature range that is optimal for each species/ecotype; and further optionally
(d) Inducing re-morphogenesis of tissues excised from a settled polyp obtained in (c), thereby obtaining a second (F2) generation in culture.

According to one embodiment the present invention concerns a method for producing coral explants (also termed spheroids) which are maintained as tissues in culture in an undeveloped form.

According to yet another embodiment, the present invention concerns a method for producing individual mature coral polyps/small colonies.

According to one embodiment said step (d) is repeated so as to obtain further generations of polyps in culture.

According to another aspect, the present invention provides coral explants which may be maintained in culture for at least several months and be induced at will to undergo re-morphogenesis into a developed polyp.

According to another aspect, the present invention provides isolated coral polyps, whereby said isolated coral polyps are obtained from a single genetic source.

According to another aspect, the present invention provides isolated coral polyps, whereby said isolated coral polyps are re-cultured from a previously cultured single genetic source.

According to another aspect, the present invention provides use of said coral explants or said coral polyps as model organisms for biological or biomedical screening. Such screening may be useful, for example, in toxicology studies of drugs, food ingredients, and cosmetics, as well as in ecotoxicology (environmental studies), and developmental biology.

The method of the invention results in the generation of small tissue explants and miniature polyps for a variety of uses with minimal space requirements. One of the greatest advantages of the method of the invention is that it uses minute amounts of natural coral tissues and thus has no detrimental impact on the donor coral population. The method of the invention produces large numbers of single polyps or tissue fragments from single genetic sources, requires neither large spaces nor ex situ culturing and provides small genetically identical polyps for uses in research, industry or as teaching tools. Moreover single genetic lines can be maintained as a bank for future uses. In addition the method of the invention can produce developed colonies/corals with or without zooxanthellae as well as with other modifications.

### BRIEF DESCRIPTION OF THE FIGURES

FIG. 1 is a photograph of an adult specimen showing mouth and peripheral region of Fungia granulosa.

FIG. 2 is a photograph of coral explants and polyps demonstrating the re-morphogenesis process. a. Explants dyed in neutral red, only the red one is viable whereas the other one is undergoing tissue deterioration. b. A callus in its early planula-like stage. c. A settled polyp—the halo around the polyp is the organic matrix. d. A settled polyp forming a mouth. e. The beginning of septae development. f. An advanced stage of re-morphogenesis, developed septae and tentacles can be observed.

FIG. 3 is a graph representing average percent survival of F. granulosa explants on different substrates for 88 days. Bars represent standard deviation, n=35.

FIG. 4 is a graph representing average percent survival of F. granulosa tissue explants on different substrates through 59 days. Bars represent standard deviation, n=40. Kaplan Meier overall comparison (substrates with and without antibiotics): p=0.035.

FIG. 5 is a graph representing average percent survival of F. granulosa explants on different substrates through 62 days. Bars represent standard deviation, n=15.

FIG. 6 is a graph representing average percent mouth development of F. granulosa tissue fragments on different substrates through 88 days. Bars represent standard deviation, n=35.

FIG. 7 is a graph representing average percent mouth development of F. granulosa explants on different substrates through 59 days. Bars represent standard deviation, n=40.

FIG. 8 is a graph representing average percent mouth development of F. granulosa explants on different substrates through 62 days. Bars represent standard deviation, n=15.

FIG. 9 is a graph representing average percent survival of F. granulosa explants in different light regimes through 9 weeks. Bars represent standard deviation, n=20.

FIG. 10 is a graph representing average percent mouth development of F. granulosa explants in different light regimes in 9 weeks. Bars represent standard deviation, n=20.

FIG. 11 is a graph representing daily temperature cycling.

FIG. 12 is a graph representing average percent survivorship of F. granulosa tissue fragments in different temperature regimes through 8 weeks. Bars represent standard deviation; n=25, p=0.001

FIG. 13 is a graph representing average percent mouth development of F. granulosa explants in different temperature regimes through 8 weeks. Bars represent standard deviation; n=25, p=0

FIG. 14 is a graph representing average percent survivorship of F. granulosa explants in two different water filtration media through 56 days. (1)=0.22 μm FSW; (2) ═0.45 μm FSW. Bars represent standard deviation; n=25, p=0.001.

FIG. 15 is a graph representing average mouth development percent of F. granulosa explants in two different water filtration media through 56 days. (1) ═0.22 μm-filtered FSW; (2)=0.45 μm-filtered FSW. Bars represent standard deviation; n=25, p=0.397.

FIG. 16 is a graph representing average percent survival of F. granulosa tissue explants from two different tissue origins through 49 days. Bars represent standard deviation, n=18, p=0.431.

FIG. 17 is a graph representing average percent mouth development of F. granulosa tissue fragments from two different tissue origins through 49 days. Bars represent standard deviation; n=18; p=0.093.

FIG. 18 is a graph representing average percent tentacle development of F. granulosa explants from two different tissue origins through 49 days. Bars represent standard deviation; n=18; p=0.051

FIG. 19 is a graph representing average percent developmental parameters of F. granulosa second-generation explants through 8 weeks. Bars represent standard deviation; n=3

FIG. 20 is a photograph of a one-year old adult polyp that is a result of a polyp culture. A. An oral view. B. An aboral view.

FIG. 21 is a photograph of Oculina patagonica development. A. An undeveloped motile callus. B. A developed polyp bearing mouth, septae and tentacles.

FIG. 22 is a photograph of A. A healthy polyp in FSW B. A bleached polyp, shown one week after adding cycloheximide.

### DETAILED DESCRIPTION OF PREFERRED EMBODIMENTS

The present invention provides novel methods for generating scleractinian coral tissue spheroids as well as functional coral polyps in vitro from differentiated coral tissue. The tissue spheroids and polyps are derived from a single genetic source and are therefore genetically identical, i.e. they may be defined as coral lines or clones.

The coral clones or lines of the invention may be used for ecotoxicological, biomedical, and developmental studies.

Tissue explants obtained by the method of the invention are not only viable, but also possess the potential to undergo full re-morphogenesis to a completely developed polyp. These tissue-originating primary polyps have good survival rates (˜20-50%). The long-term survival of the clones (for over 3 months in tissue grade state and for over one year as polyps in culture) provides a basis for their usefulness in short or long term coral studies.

### Terms and Definitions:

As used herein, the term “Scleractinia” refers to “stony” corals which are exclusively marine animals comprising soft tissue and a hard skeleton.

As used herein, the term “ecotype” refers to a distinct breed of organisms that is closely linked in its characteristics to the ecological surroundings it inhabits.

As used herein, the term “coral tissue” refers to soft tissue of Scleractinia corals comprising three layers: an outer epidermis (the embryonal ectodermal layer), a gastrodermal layer (the embryonal endoderm layer), and between them a mesoglea.

The term “aragonite skeleton” refers to the rigid scleractinian skeleton, which lies external to the polyps that make it, and is composed of calcium carbonate in the crystal form aragonite.

As used herein, the term “coral tissue fragment” refers to a fragment which includes ectoderm, endoderm, and mesoglea, but is devoid of skeletal tissue.

As used herein, the terms “spheroid” “callus” and “explant” are used interchangeably and refer to coral a tissue fragment which is maintained viable in culture in an undeveloped form, i.e. it does not evolve into a polyp.

As used herein, the term “polyp” refers to a coral, having a roughly cylindrical body and an oral opening usually surrounded by tentacles.

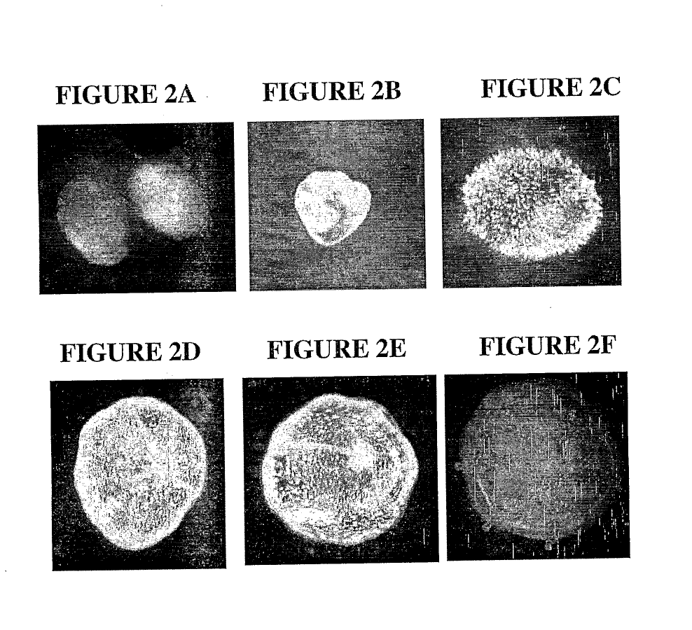

As used herein, the term “re-morphogenesis” refers to a development process in which the spheroid reorganizes its body form into a polyp by developing mouth, septae and tentacles (see FIG. 2c-f) and thus a polyp is formed in culture.

As used herein, the terms “seawater”, “seawater media” or “seawater solutions” are used interchangeably to denote media or solutions having seawater properties. Seawater, i.e. the water of the sea, is distinguished from freshwater by its appreciable salinity. This salinity is mainly achieved due to the presence of sodium and chloride ions, however certain trace elements e.g. magnesium, calcium and potassium are also present. Seawater may be obtained from a sea or produced artificially by reconstitution of the seawater content, i.e. by supplementing fresh water with ions (“artificial seawater”). In the context of the present invention, the concentration of the ions in the “seawater”, “seawater media” or “seawater solutions” may be adjusted according to the culture requirement e.g. the amount of calcium, chloride, magnesium etc may be increased or reduced. In addition, certain modifications may also be made in the seawater pH.

As used herein, the term “toxicology” refers to the study of the adverse effects of chemical and physical agents on living organisms.

Excision of Coral Tissue Fragments from Polyps and Cultivation of Viable Coral Tissue Explants

Coral tissue fragments are obtained from adult corals and excised into pieces of approximately 1-3 mm³ using sterile instruments, such as fine tweezers (no. 5 dumont), and aseptic techniques. Immersion of corals in a modified seawater based solution (e.g. calcium free seawater) for up to 6 hours can also be used in several species (Faviids or Pocillopora) in order to assist in the release of tissues from the skeleton. Following this the tissues are rinsed a number of times in filtered or artificial seawater.

The pieces of tissue are then transferred into sterilized tissue culture solution in sterile vessels. Preferably said tissue culture solution comprises seawater. The seawater may either be filtered natural seawater or artificial seawater which is commercially available. The seawater includes Ca²⁺ and Mg²⁺.

Following formation of tissue spheroids (explants) (between 2-4 days), the spheroids are transferred to sterile culture vessels containing a culture solution, preferably seawater. Spheroid growth rates can be enhanced by supplementing the culture with optimal intensities of photosynthetic photon flux densities of approximately 20-30 μmol/m² s.

Culturing of viable coral spheroids can be carried out in complete darkness, but this requires a specific supplemental formula to the culture solution, since in complete darkness algae survival is compromised and an external source of food is required, e.g. amino acid preparations or Artemia.

### Maintenance of Tissue Spheroids

Maintenance of coral spheroid cultures should be within the optimal temperature range for the species, with periodic changes of tissue culture solution. This may be experimentally determined for each ecotype or genotype within a specific environment. For example, the optimal temperature for maintenance of a spheroid of the Red Sea, e.g. F granulosa is about 19° C. to about 21° C. The temperature is preferably not higher than about 22° C. for this species/genotype, as in higher temperatures the spheroids will be induced to undergo re-morphogenesis. For coral species which originate from seas having a cooler lower range of water temperature e.g. the Mediterranean Sea, a lower temperature can be used, e.g. about 16° C. for Oculina patagonica. Coral species or ecotypes may also be obtained from cold sea environments typical for example to deep-sea waters, at which case even colder temperatures may be used for maintenance.

### Induction of Re-Morphogenesis

To induce polyp development, tissue spheroids are maintained at an optimal culturing temperature for each species/ecotype (for example for Red Sea F. granulosa temperature range of about 22° C. to about 30° C.) and are subjected to the following protocol:
(1) One week after tissue excision from the polyp, ⅓-½ of the culture solution is carefully removed and new culture solution is added so that the volume remains unchanged.
(2) Culture solution is refreshed every 7-14 days as described above, while avoiding mechanical disruption of the contact between the tissue spheroid and the culture vessel surface.
(3) Once the tissue explants have settled on the culture vessels (which occurs about 7 days or more after excision), the vessels are cleaned of any algal, bacterial, or invertebrate fouling by wiping the surfaces, such as by using a sterile nylon no 2 paintbrush.

These protocols, including filtration, are preferably carried out in glassware or other chemically inert material.

(4) Once a mouth, septae and tentacles develop (a mouth-about two weeks after settlement, septae-after about one more week and tentacles after about another week, and in parallel skeleton deposition commences) the culture vessels are transferred to a water table or larger culturing vessel. The polyps are fed weekly with Artemia nauplii (1-day following hatching) or bryozoan recipe, or any suitable coral food known to a person skilled in the art. Following the feeding the water is changed and the vessel surfaces are kept clean.

The cultures are maintained under optimal temperature conditions for that coral species, for example as determined by the temperature of the sea from which the coral species is obtained. The “Red Sea” being an example of warm temperature conditions, e.g. 22-30° C. while the Mediterranean Sea being an example of cool temperature conditions e.g. 16-30° C.

The process of culturing the biopsies creates tissue that follows polyp genesis. Using this process, reorganization of the two primary tissue types occurs. This is followed by invagination and settlement of the coral tissue mass. Settlement is followed by primary structure formation, including the oral invagination, septae, and tentacles. Ultimately, the polyp deposits aragonite skeleton and grows. The protocol is configured for mass tissue culturing of over a hundred biopsies taken from a single coral polyp source (i.e. single genetic source) and can be then harvested with or without an aragonite skeleton.

Moreover, the re-morphognesis can repeat itself as second generation of polyp cultures can be obtained in accordance with the invention. Thus suggesting line-characteristics for the coral tissues obtained in accordance with the invention. The adult polyps that the tissues were extracted from were maintained in the lab for over a year. This suggests that this method can also be successfully used in aquaculture as well as in biological studies.

The formation of zooxanthellae-free polyps can be used in bleaching studies. Bleaching causes great concern worldwide (Goreau and Hayes 1994, Brown 1997, Hoegh-Guldberg 1999). Bleaching can be achieved by using chemical means, e.g. antimycotics or antibiotics, e.g. cycloheximide.

The method of the invention is suitable for culturing scleractinian corals, including but not limited to the solitary coral Fungia granulosa and, and the colonial corals Favia favus and Oculina patagonica.

### Materials and Methods

### Tissue Excision

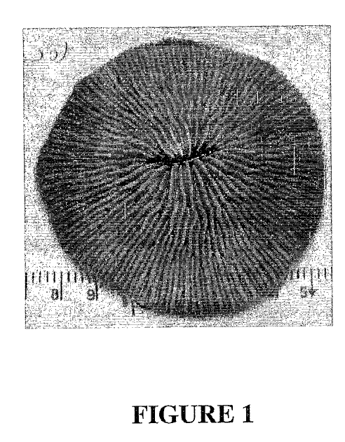

Tissue from the Red Sea coral Fungia granulosa was removed mechanically using fine tweezers. The tissues were taken from the mouth region (M) or from the peripheral (P) region of the coral polyp (FIG. 1). Ten to thirty tissue explants were transferred via a number of washes in 22 μm FSW (filtered sea water) and then placed in 3-12 cm Petri dishes filled with 22 μm FSW for a period of 24 hours until tissue rounding (callus formation) was evident.

In order to minimize possible infections by mucus associated microorganisms surface mucus was removed from the corals prior to tissue excision, by placing them on a funnel and allowing the mucus to drip for 20 minutes. The corals were then returned to an aquarium with filtered seawater, and allowed to recuperate for two days prior to removal of tissue.

### Neutral Red Vital Staining Determination of Tissue Viability

In order to test viability of the tissue explants a neutral red assay was performed (Weeks and Svendsen 1996, Stachowicz and Hay 1999). Tissues were maintained at a temperature of 24° C. and under 20 μmol/m² s of light for 10 days. The tissues were then placed in a solution of neutral red, diluted in 0.22 μm FSW (0.57 g/l) for 10 minutes. The tissues were washed in FSW and their viability was shown. Tissues that incorporate the dye are viable tissues while those that do not are moribund (Weeks and Svendsen 1996, Stachowicz and Hay 1999).

### Maintenance of Cultured Polyps

After the formation of polyps from calluses or explants, the polyps were transferred into an aquarium containing seawater and aeration, or put in a closed water flow system. Polyps were then fed weekly with Artemia nauplii following which water was replaced (natural or artificial sea water) if necessary. Polyps were maintained under commercially available T5 fluorescent lights (white and blue spectra) or natural sunlight.

### F2—Second Generation of Polyp Culture

Tissue was excised from two 10-month old cultured polyps that had been maintained in an aquarium (see tissue origin experimental conditions). After forming calluses the F2 were placed in glass Petri dishes filled with 0.45 μm FSW, in 32 μmol/m² s of light and a daily temperature cycle of 23-30° C. In addition, in order to activate swift release of fungiid polyps from their substrate and from their stalks, the explants were maintained in two light regimes, high (130 μmmol/m² s) and low light (20 mol/m² s).

Favia favus Polyp Culture

Tissue from the Red Sea colonial coral Favia favus was removed mechanically using fine tweezers. Tissues were rinsed in filtered natural seawater (0.22 μm FSW) placed in glass Petri dishes 24 hours after removal. The tissues were maintained under the same conditions as the fungiid corals.

Oculina patagonica Polyp Culture

Tissue from the Mediterranean coral Oculina patagonica was removed mechanically using fine tweezers. Tissues were rinsed in filtered natural seawater (0.22 μm FSW) placed in glass Petri dishes 24 hours after removal. The tissues were maintained under the same conditions as the fungiid corals.

### Modified Polyp Culture

Polyps were transferred into different concentrations of the fungicide cycloheximide (SIGMA cat no: 01811) (10 mg/1, 20 mg/l and 28 mg/l) for a period of one month. Polyps were placed under 20 μmol/m² s of light and a daily temperature of 25° C.

### EXAMPLES

Using fine tweezers tissue fragments were explanted from an adult coral Fungia granulosa that had been fragmented using a hammer and clean chisel. Approximately 24 hours after explanting, the tissues rounded up into a planula-like morphology (see FIG. 2a) and became very motile. In order to determine viability of the tissues a neutral red viable staining test was performed. As shown in FIG. 2a the live tissues were dyed red, whereas the dead or disintegrated ones did not take in the dye. The tissue explants, which can also be referred to as calluses, were maintained in this form for up to three months when the water temperature was low (˜19° C.).

The explants were not only viable, but also showed the potential of becoming a fully-grown polyp. When maintained in the proper conditions, the callus or explant settles and develops a mouth, septae and tentacles (see FIG. 2c-f). This process is referred to as re-morphogenesis in which a tissue from an adult polyp reorganizes its body form into a new polyp. This process in the optimal conditions occurs within a month: settling after a week, forming a mouth after two weeks, forming septae after three weeks and tentacles after four weeks.

The optimal protocol for maintaining this polyp culture was determined after a series of experiments. The main parameters that were examined are survivorship of the explants (or polyps in the later stages) or mouth development—a stage which represents the turning point in which an explant or callus becomes a polyp.

Experiments were performed to determine the optimal conditions for survivorship and development (formation of mouth as a characteristic of polyp formation) of the tissues. The survivorship parameters refer to tissue survivorship without taking into account if the tissues developed into polyps or remained at tissue grade stage. This parameter was used to establish optimal conditions for primary stages of tissue or polyp culture. On the other hand, mouth formation is a characteristic of re-morphogenesis and therefore the establishment of polyp culture.

### Determination of Optimal Conditions for Survivorship and Development

The following parameters were examined:

### Substrate

I. General: In order to examine the effect of the substrate, excised tissues of approximately the same size were placed in Petri dishes with 7 different substrata. In each experiment the fragments were maintained at 23° C. under constant light and examined daily for settlement. This experiment was concluded after three months.

The following substrates were used:
1) Glass Petri dish—sterilized in an autoclave
2) Scratched—autoclaved glass

### 3) Plastic

4) Scratched plastic
5) Tissue culture plates
6) Coral skeleton fragments. Skeleton fragments were crushed using a hammer and sterilized in an autoclave. They were then glued to a plastic Petri dish using super glue and rinsed three times in DDW and once in FSW.
7) Mesoglea strips. Mesoglea strips (excised from the bell of Rhopilema nomadica, class: Scyphzoa) were rinsed three times in DDW and were placed in a plastic Petri dish with FSW.

II. Substrate and antibiotics: In order to determine whether antibiotics have an effect on the survival of the tissues, tissue explants or spheroids were placed on 4 different substrates (sterile glass Petri dishes, sterile scratched glass, plastic and scratched plastic) in FSW or FSW+antibiotics (0.5 mg/ml kanamycin and penicillin G) SIGMA cat no. N2889.

The tissues were maintained at a constant temperature of 23° C. for two months under constant light.

III. Transparencies: Polyester transparency films were used in order to assess if tissue would settle on substrate that could be easily cut and manipulated. For sterility the transparencies were soaked in 70% ethanol for 24 hours, washed in FSW before being placed inside plastic Petri dishes. Growth on transparencies was compared with growth on other substrates i.e. plastic and glass.

### Light Intensity

Glass Petri dishes containing tissue explants were placed under four different light regimes: high light (106 μmmol/m² s), medium light (85 μmol/m² s), low light (22 μmol/m² s), and dark (2.5 μmol/m² s). In the first three weeks of the experiment the tissues were maintained under 12:12 light/dark regime, which was then changed to 9 hours light: 15 hours dark (due to polyps bleaching at high light intensities). In all experiments the temperature regime was 26° C. during the day and 23° C. during the night.

### Temperature: Constant Versus Cycling Temperatures

Two different temperature regimes were used—constant temperature (25° C.) and a cycling of daily temperature (23-30° C.). Both regimes used white and blue light; however the constant temperature was under 20 μmol/m² s of light and the cycling under 32 μmmol/m² s of light.

### Tissue Source and State

Tissues were separated to mouth region (1 cm away from parent polyp mouth) and peripheral region. The resulting tissue explants were placed in glass Petri dishes filled with FSW under ambient light conditions and under a diurnal temperature cycle of 20-28° C. Some explants were maintained at low temperatures (19° C.) and monitored for morphological changes.

### Water Source and Filtration

Tissues were separated to mouth region and peripheral region as above. Tissue explants from each tissue type were placed in glass Petri dishes filled with FSW filtered with 0.22 μm pore filter or 0.45 μm pore filter, under ambient light and an average diurnal temperature cycle of 20-28° C., or in artificial seawater (produced from commercially available sea salt).

The percent of explants with the characteristic in question (survivorship or mouth development) was counted in each dish within a treatment. It is noted that most of the explants that developed mouths survived and developed into polyps. The scoring was calculated by averaging the measured percentages. A Meier-Kaplan Survivorship curve (Kaplan and Meier, 1958) was developed and a Cox—Mantel Log rank test was carried out (see http://www.medcalc.be/index.php).

### Substrate

### Survivorship

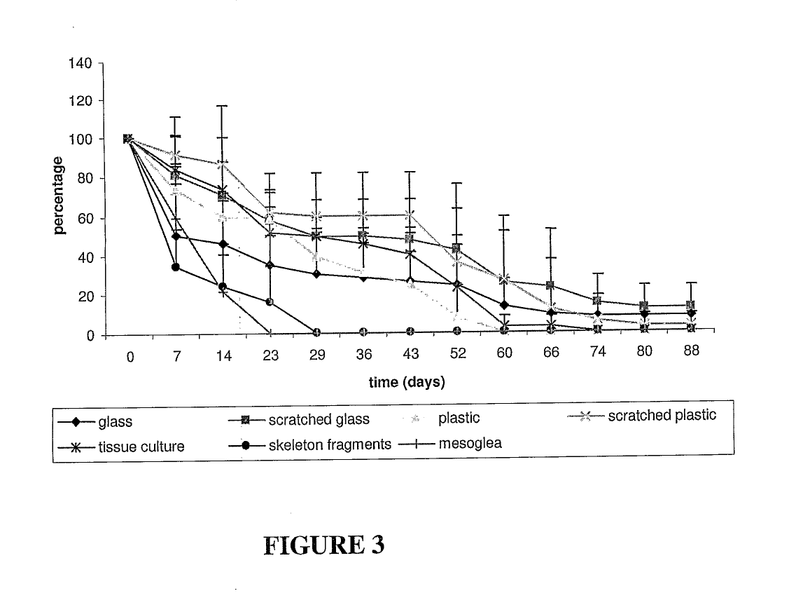

According to the Kaplan Meier survival test the longest survival times in the first experiment were in the scratched plastic and scratched glass (see table 2). The Cox Mental tests shown in table 1, indicate that explants on the scratched substrates showed significantly higher survival rates than those on the non-scratched substrates (p<0.05). According to FIG. 3, the scratched glass shows the highest average percent survival compared to all other substrates. The lowest survivorship was demonstrated in the mesoglea and skeleton fragments substrates compared to all the other substrates in this experiment (Table 1 p<0.05), and therefore were not used again.

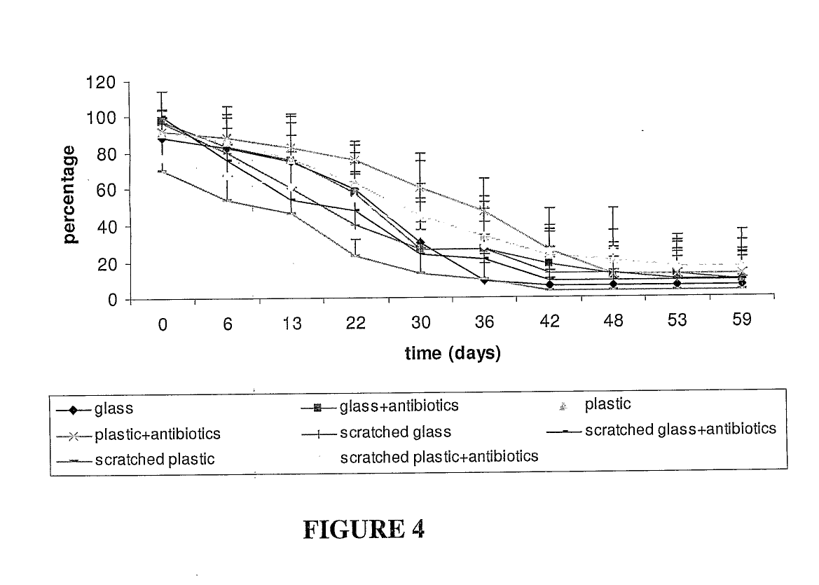

A second experiment was performed using four of the substrates included in the first experiment, with a supplement of antibiotics in order to examine if antibiotics may have an effect on the survival of the tissues. According to the Kaplan Meier overall comparison test, a significant difference was found (p<0.05) in survivorship between explants in antibiotics and those without antibiotics, suggesting that antibiotics has a positive effect on the survival of the tissues. Interestingly in this experiment, the scratched substrates did not appear to be the best substrates for survival. According to FIG. 4 and Table 4 the substrate that shows the highest survival rates and survival time is plastic (see Table 4), there is a significant difference between plastic and all the other substrates (see Table 3).

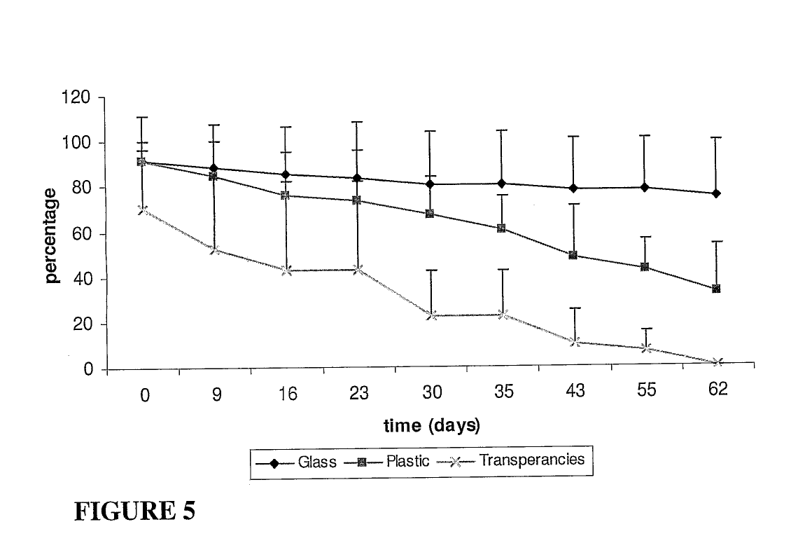

A third experiment was performed using glass and plastic substrates. In addition plastic transparencies were added as a substrate to assess the usefulness in providing a substrate which is easy to manipulate. The high survival percentage was found in explants cultured in the glass plates compared to the other substrates. There is a significant difference between glass and the other substrates (see Table 5 table 6, FIG. 5). Transperancies proved to be ineffective and none of the explants survived by the end of the experiment.

TABLE 1
Cox-Mantel tests of survival rates of the F. granulosa explants on different
substrates

| | | | | | tissue | | |
|---|---|---|---|---|---|---|---|
| | | scratched | | scratched | culture | skeleton | |
| substrate | glass | glass | plastic | plastic | plates | fragments | mesoglea |
| Glass | — | p = 0.0015 | p = 0.8072 | p = 0.0008 | p = 0.1803 | p = 0.0001 | p = 0.0004 |
| Scratched | | — | p = 0.0006 | p = 0.8784 | p = 0.0628 | p = 0 | p = 0 |
| glass | | | | | | | |
| Plastic | | | — | p = 0.0003 | p = 0.1111 | p = 0.0002 | p = 0.0009 |
| Scratched | | | | — | p = 0.0408 | p = 0 | p = 0 |
| plastic | | | | | | | |
| Tissue | | | | | — | p = 0 | p = 0 |
| culture | | | | | | | |
| plates | | | | | | | |
| Skeleton | | | | | | — | p = 0.8643 |
| fragments | | | | | | | |
| Mesoglea | | | | | | | — |

TABLE 2
Average survival time of F. granulosa explants on each substrate
(experimental period of 88 days)

| | Average survival |
|---|---|
| Substrate | time |
| 1. glass | 56.33 ± 1.09 |
| 2. scratched glass | 62.30 ± 1.19 |
| 3. plastic | 57.49 ± 1.03 |
| 4. scratched plastic | 63.72 ± 1.12 |
| 5. tissue culture | 60.35 ± 1.09 |
| plates | |
| 6. skeleton | 50.13 ± 1.10 |
| fragments | |
| 7. mesoglea | 50.65 ± 1.26 |

TABLE 3
Mantel-Cox test of survival rates of the F. granulosa explants -
comparison between different substrates

| | | | | Scratched |
|---|---|---|---|---|
| Substrate | Glass | Plastic | Scratched glass | plastic |
| 1. Glass | — | p = 0.003 | p = 0.601 | p = 0.07 |
| 2. Plastic | | — | p = 0.001 | p = 0 |
| 3. Scratched glass | | | — | p = 0.218 |
| 4. Scratched plastic | | | | — |

TABLE 4
Average survival time of F. granulosa explants on each substrate
(experimental period of 59 days)

| | average survival |
|---|---|
| substrate | time |
| 1. glass | 42.84 ± 0.607 |
| 2. plastic | 45.24 ± 0.62 |
| 3. scratched glass | 41.98 ± 0.66 |
| 4. scratched plastic | 39.94 ± 0.73 |

TABLE 5
Cox-Mantel tests of survival rates of the
F. granulosa explants - comparison between different substrates

| substrate | 1. glass | 2. plastic | 3. transparencies |
|---|---|---|---|
| 1. glass | — | p = 0 | p = 0 |
| 2. plastic | | — | p = 0 |
| 3. transparencies | | | — |

TABLE 6
Average survival time of F. granulosa explants on each substrate
(experimental period of 62 days)

| | Average survival |
|---|---|
| substrate | time |
| 1. glass | 53.47 ± 1.03 |
| 2. plastic | 48.84 ± 1.01 |
| 3. transparencies | 38.90 ± 1.15 |

### Mouth Development

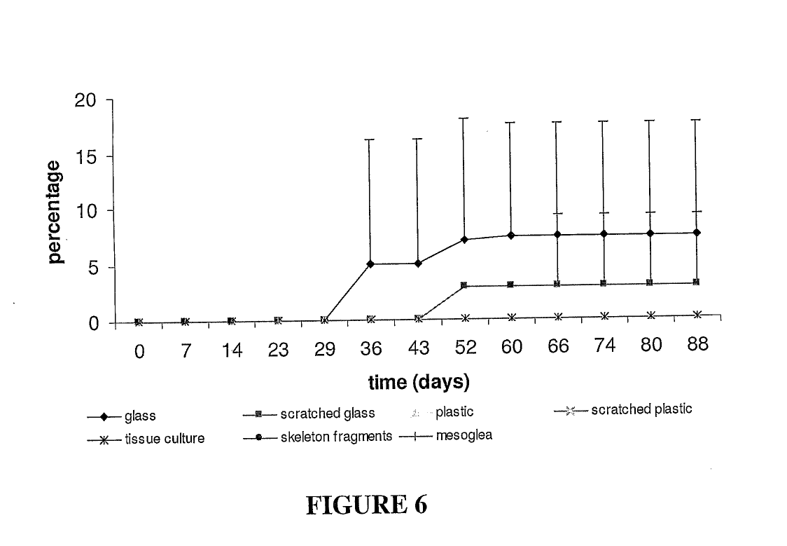

In the first experiment only the explants cultured on glass or scratched glass plates developed mouths (see FIG. 6). In addition in the glass substrate there were significantly more polyps that developed mouths than in the scratched glass (Table 7, p<0.05). Furthermore, in the glass substrate the mouth development time was shorter than in all other substrates (see Table 8).

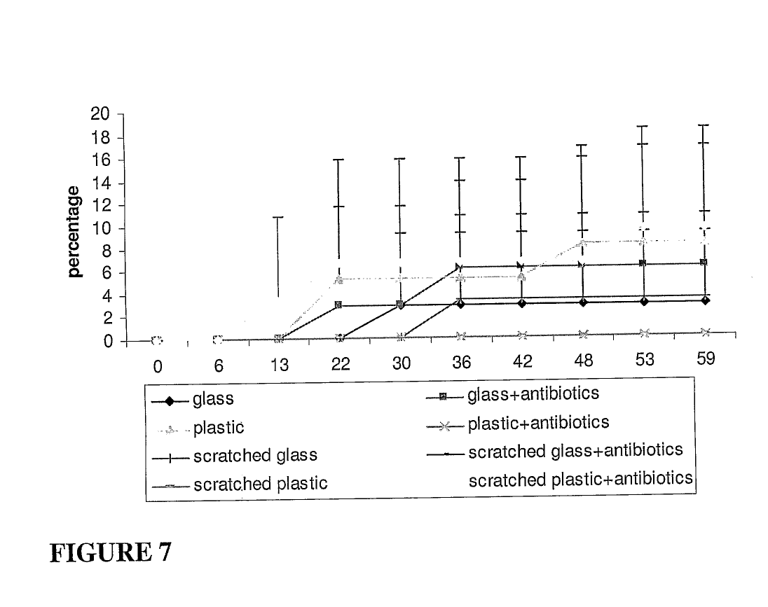

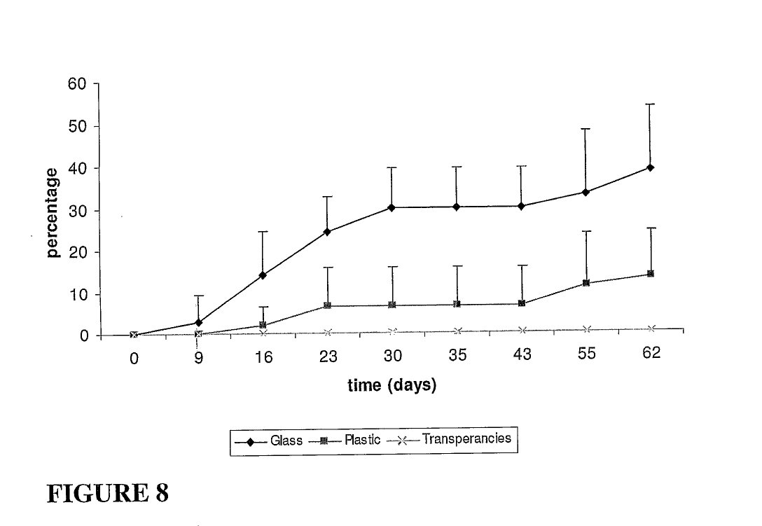

In the second experiment, tissues in all substrates developed mouths, however in low percentages and only following a long period of time (see FIG. 7 and Table 10). A supplement of antibiotics was used in order to examine if it had an effect on the mouth development, thus affecting the rates of transformation into polyps. According to the Cox-Mantel comparison test, no significant difference was shown (p>0.05) between treatments, suggesting that antibiotics have no effect on the rates of mouth development. FIG. 7 shows, however, that the highest mouth development percentage is in the scratched plastic+antibiotics substrate. The scratched glass showed a significant difference compared to all substrates and took the longest to develop mouths (see Table 9, 10). In the third experiment, there was no mouth development at all on the transparencies since none of the explants survived (FIG. 8). The highest rates of mouth development and the shortest amount of time until development was observed in explants grown on the glass substrate, with a significant difference compared to plastic (see Table 11, 12). Overall, it appears that glass is the most effective substrate in terms of mouth development.

TABLE 7
Cox-Mantel tests of mouth development of the F. granulosa explants -
comparison between different substrates

| | | scratched | | scratched | skeleton | |
|---|---|---|---|---|---|---|
| substrate | glass | glass | plastic | plastic | fragments | mesoglea |
| Glass | — | p = 0.0008 | p = 0 | p = 0 | p = 0 | p = 0 |
| Scratched | | — | p = 0.0063 | p = 0.0143 | p = 0.0083 | p = 0.0227 |
| glass | | | | | | |
| Plastic | | | — | N.D. | N.D. | N.D. |
| Scratched | | | | — | N.D. | N.D. |
| plastic | | | | | | |
| Skeleton | | | | | — | N.D. |
| fragments | | | | | | |
| Mesoglea | | | | | | — |

TABLE 8
Average time until mouth development of F. granulosa explants on each
substrate (in 88 days)

| | average time until mouth |
|---|---|
| substrate | development |
| Glass | 85.09 ± 0.56 |
| Scratched glass | 87.31 ± 0.34 |
| Plastic | — |
| Scratched plastic | — |
| Skeleton fragments | — |
| Mesoglea | — |

TABLE 9
Cox-Mantel tests of mouth development rates of the
F. granulosa explants - comparison between different substrates

| | | | 3. scratched | 4. scratched |
|---|---|---|---|---|
| substrate | 1. glass | 2. plastic | glass | plastic |
| 1. glass | — | p = 0.937 | p = 0.012 | p = 0.847 |
| 2. plastic | | — | p = 0.022 | p = 0.818 |
| 3. scratched | | | — | p = 0.007 |
| glass | | | | |
| 4. scratched | | | | — |
| plastic | | | | |

TABLE 10
Average time until mouth development of F. granulosa
explants on each substrate (in 59 days)

| | average time until mouth |
|---|---|
| substrate | development |
| 1. glass | 58.12 ± 0.23 |
| 2. plastic | 58.14 ± 0.24 |
| 3. scratched glass | 58.75 ± 0.14 |
| 4. scratched | 58.03 ± 0.27 |
| plastic | |

TABLE 11
Cox-Mantel test of mouth development rates of the
F. granulosa explants - comparison between different substrates

| substrate | 1. glass | 2. plastic | 3. transparencies |
|---|---|---|---|
| 1. glass | — | p = 0 | p = 0 |
| 2. plastic | | — | p = 0 |
| 3. transparencies | | | — |

TABLE 12
Average time until mouth development of F. granulosa
explants on each substrate (in 62 days)

| | average time until mouth |
|---|---|
| substrate | development |
| 1. glass | 53.25 ± 0.99 |
| 2. plastic | 59.64 ± 0.56 |
| 3. transparencies | — |

### Artificial Light

### Survivorship

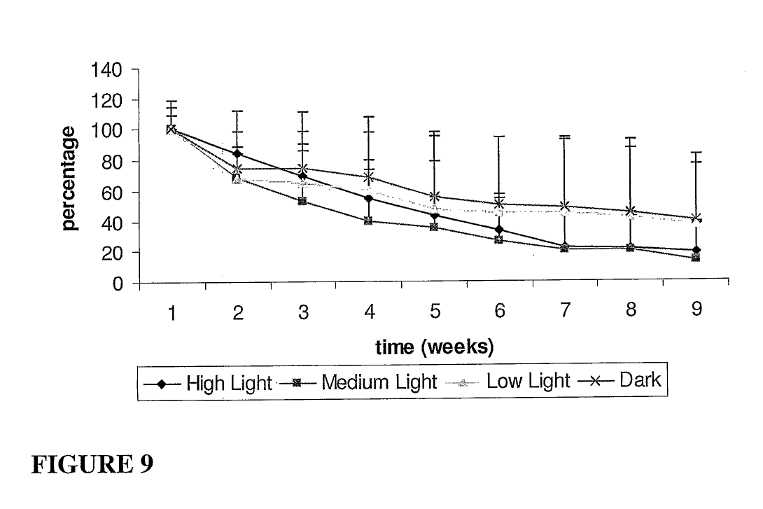

In this experiment, the most effective light regime for tissue survival was tested. FIG. 9 and Table 13 show that there is a significantly higher survival percentage under the dark and low light regimes. The highest average survival time (see Table 14) was under the low light regime. Interestingly it is evident that high light showed significantly better results than medium light (see Table 13, 14).

TABLE 13
Cox-Mantel tests of survival rates of the F. granulosa tissue
explants - comparison between different light regimes

| | | | | |
|---|---|---|---|---|
| Light regime | 1. high | 2. medium | 3. low | 4. dark |

| | | | |
|---|---|---|---|
| 1. high | p = 0.037 | p = 0.004 | p = 0.216 |
| 2. medium | | p = 0 | p = 0.323 |
| 3. low | | | p = 0 |
| 4. dark | | | |

TABLE 14
Average survival time of F. granulosa explants
in each light regime (examined after 9 weeks)

| Light regime | average survival time |
|---|---|
| 1. high | 7.060 ± 0.108 |
| 2. medium | 6.725 ± 0.095 |
| 3. low | 7.379 ± 0.096 |
| 4. dark | 6.850 ± 0.09 |

### Mouth Development

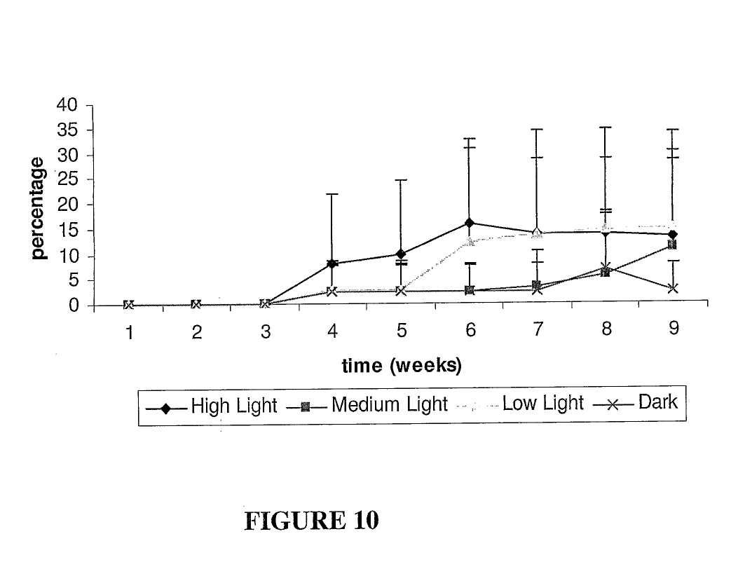

In this experiment, the most effective light regime for mouth development was tested. As can be seen in table 16, there was a significant difference between all light regimes except between low and medium light. Mouths developed fastest in the high light regime (see Table 15). However there was some mortality in the high light regime, resulting in a lower percentage of explants with mouth at the end of the experiment (FIG. 10). A more successful light regime therefore is the low light regime that shows high percentage of mouth development, which remains persistent throughout the experiment.

TABLE 15
Cox-Mantel tests of mouth development rates of the F. granulosa
explants - comparison between different light regimes

| | | | | |
|---|---|---|---|---|
| Light regime | 1. high | 2. medium | 3. low | 4. dark |

| | | | |
|---|---|---|---|
| 1. high | p = 0 | p = 0.017 | p = 0 |
| 2. medium | | p = 0.075 | p = 0 |
| 3. low | | | p = 0 |
| 4. dark | | | |

TABLE 16
Average time until mouth development of F. granulosa explants
in each light regime (after 9 weeks)

| | average time in weeks until mouth |
|---|---|
| Light regime | development |
| 1. high | 8.432 ± 0.079 |
| 2. medium | 8.803 ± 0.041 |
| 3. low | 8.685 ± 0.051 |
| 4. dark | 8.930 ± 0.025 |

### Temperature

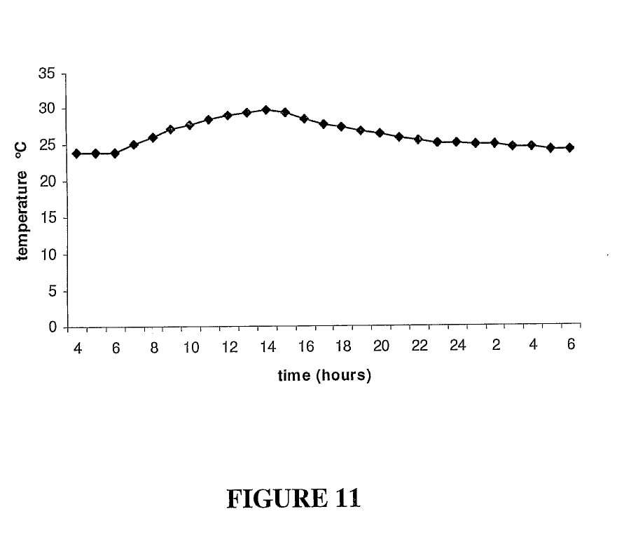

In this experiment survivorship and mouth development were tested at a constant daily temperature or at a cycling daily temperature (FIG. 11).

### Survivorship

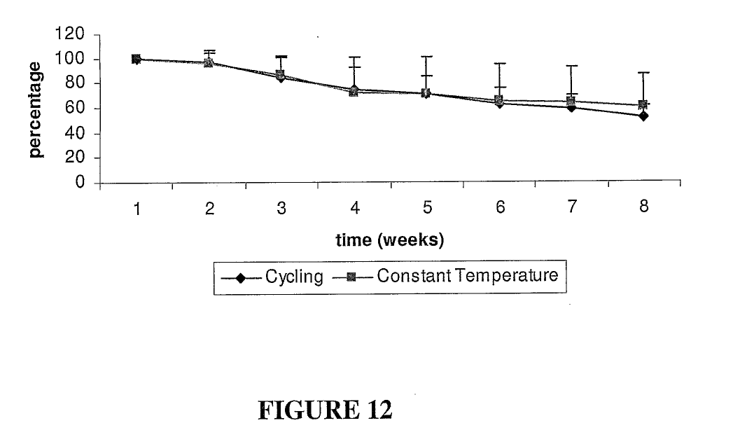

According to FIG. 12 and Table 17, constant temperature showed higher rates of survivorship and higher survival time, with significant differences between the temperature regimes (p<0.05).

TABLE 17
Average survivorship time of F. granulosa explants
in each temperature regime (in 8 weeks)

| Temperature | average survival time in weeks |
|---|---|
| 1. cycling | 6.983 ± 0.041 |
| 2. constant | 7.140 ± 0.041 |
| temperature | |

### Mouth Development

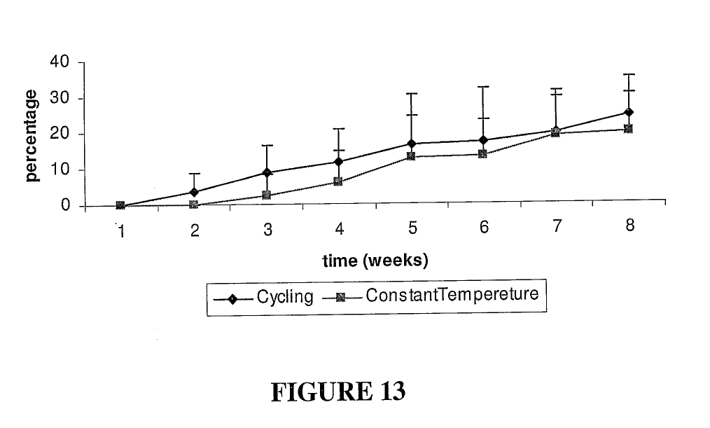

The cycling showed higher rates of mouth development and shorter development time (see FIG. 13 and Table 18), with a significant difference between the temperature regimes (p<0.05).

TABLE 18
Average time until mouth development of F. granulosa
exlants in each temperature regime (in 8 weeks)

| | Average time in weeks until |
|---|---|
| Temperature | mouth development |
| 1.cycling | 7.515 ± 0.032 |
| 2.constant | 7.696 ± 0.027 |
| temperature | |

### Water Filtration

### Survivorship

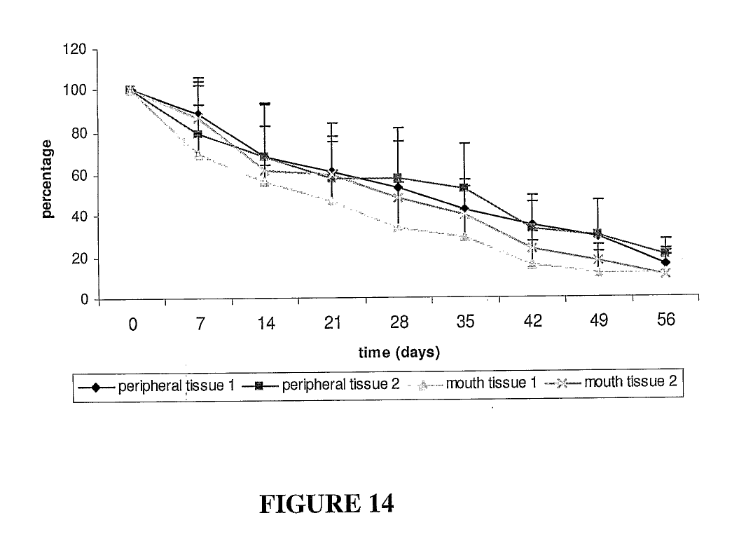

Two different seawater filtration protocols were examined in order to test their influence on the survivorship of the tissues. 0.45 μm filtered FSW showed higher rates of survivorship with a significant difference from 0.22 μm-filtered FSW (p<0.01, see FIG. 14).

TABLE 19
Average survivorship time of F. granulosa explants in
each temperature regime (in 56 days)

| Tissue origin | | average survival time |
|---|---|---|
| 0.22 μm | 1. peripheral | 7.123 ± 0.072 |
| FSW | tissue | |
| | 2. mouth tissue | 6.458 ± 0.078 |
| 0.45 μm | 1. peripheral | 7.142 ± 0.079 |
| FSW | tissue | |
| | 2. mouth tissue | 7.011 ± 0.087 |

### Mouth Development

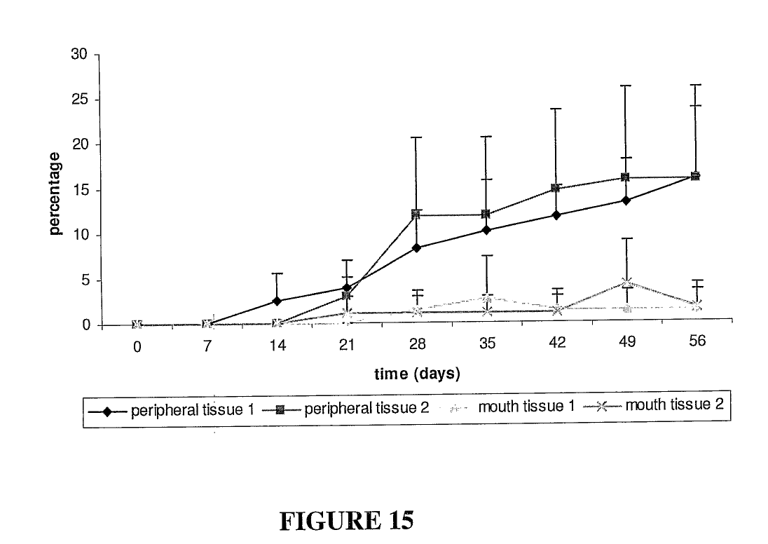

Water filtration showed no effect on the development of mouths, thus no effect on the development of polyps (p>0.05, see FIG. 15 and Table 20).

TABLE 20
Average time until mouth development of F. granulosa
explants in each temperature regime (in 56 days)

| | | Average time until mouth |
|---|---|---|
| Tissue origin | | development |
| 0.22 mm | 1. peripheral | 8.673 ± 0.041 |
| FSW | tissue | |
| | 2. mouth | 8.967 ± 0.016 |
| | tissue | |
| 0.45 mm | 1. peripheral | 8.640 ± 0.045 |
| FSW | tissue | |
| | 2. mouth | 8.943 ± 0.023 |
| | tissue | |

Artificial seawater was also tested and explants were found to be viable, settled and developed into polyps.

### Tissue Origin

### Survivorship

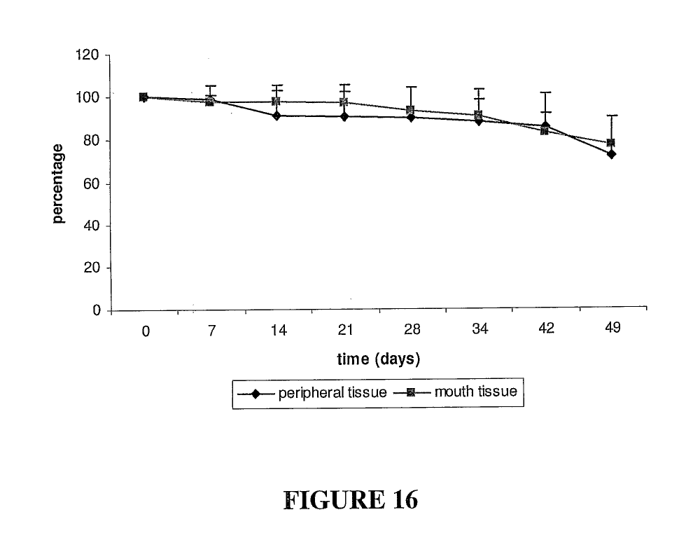

The effect of tissue origin from the adult polyp was examined in terms of survivorship. No significant difference between the two tissue origins (mouth tissue and peripheral tissue) was shown (see FIG. 16), and they had very similar survival time (see Table 21).

TABLE 21
Average survival time of F. granulosa exlants
from two different tissue origins (after 49 days)

| Tissue origin | average survival time |
|---|---|
| 1. peripheral tissue | 46.459 ± 0.210 |
| 2. mouth tissue | 46.684 ± 0.239 |

### Mouth Development

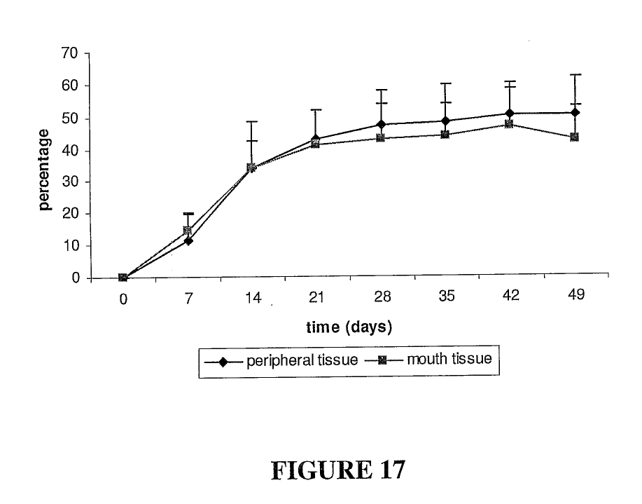

The effect of tissue origin from the adult polyp was examined in terms of mouth development. No significant difference between the two origins was shown (see FIG. 17), and the time until mouth development was very similar (see Table 22).

TABLE 22
Average time until mouth development of F. granulosa
explants from two different tissue origins (after 49 days)

| | Average time until mouth |
|---|---|
| Tissue origin | development |
| 1. peripheral tissue | 39.287 ± 0.325 |
| 2. mouth tissue | 39.733 ± 0.388 |

### Tentacle Development

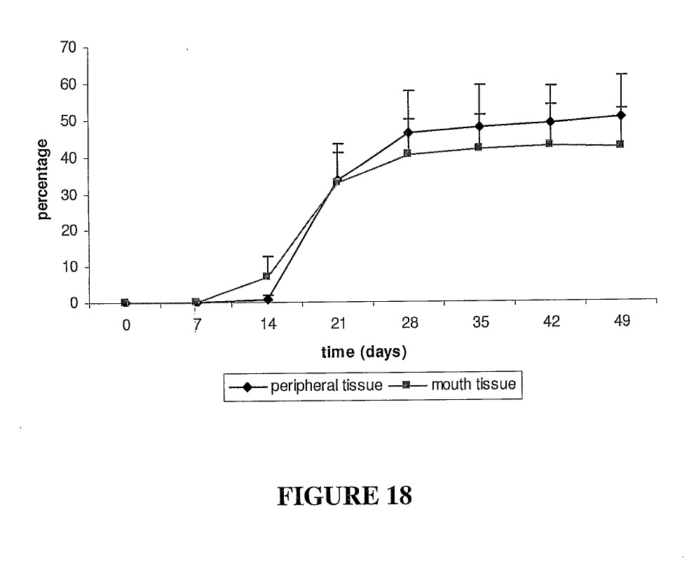

The effect of tissue origin was also examined in terms of tentacle development. No significant difference between the two origins was shown (see FIG. 18), and they had very similar survival time (see Table 23).

TABLE 23
Average tentacle development of F. granulosa
explants from two different tissue origins (in 49 days)

| | Average time until tentacle |
|---|---|
| Tissue origin | development |
| 1. peripheral | 41.703 ± 0.282 |
| tissue | |
| 2. mouth tissue | 42.181 ± 0.336 |

Cultured fungiid polyps develop on a short stalk attached to the glass Petri dish in the aquarium. Following release from the substrate, the polyp detaches from the stalk and the stalk develops into an additional polyp. In order to activate swift release of fungiid polyps from their substrate and from their stalks, the high surface light regime (130 μmmol/m² s) was used and resulted in faster release than the low regime.

### F2—Second Generation of Polyp Culture

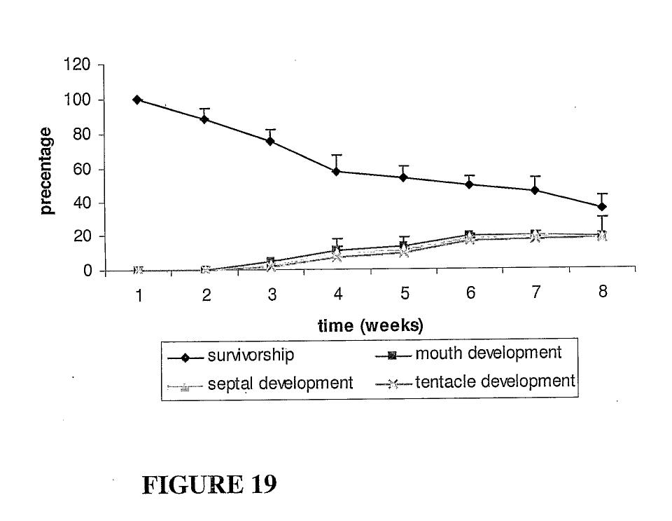

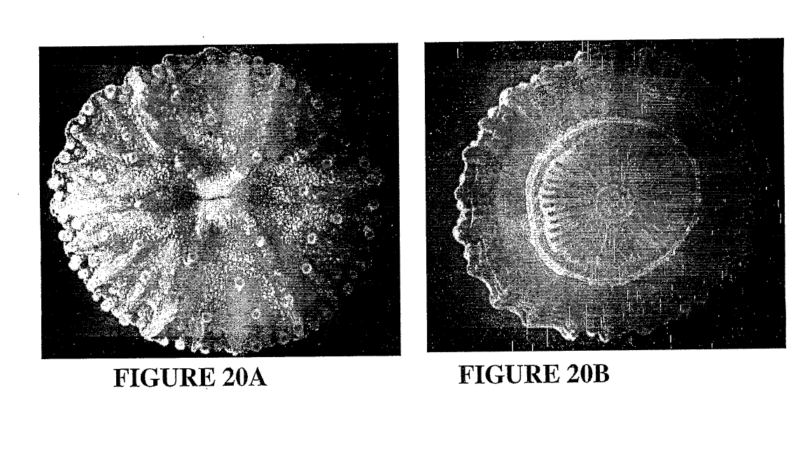

To test the possibility of cultivating a second generation of tissues or polyps in culture, tissues were explanted from 10 month old adult polyps that had been previously cultivated in the lab (see FIG. 19 and FIG. 20). Mouth development reached 19% and septal and tentacle development reached 18% by week 8. Developmental parameters show that mouth, septae and tentacles start to develop in the third week.

Favia favus Polyp Culture

In order to examine the ability of other species to undergo re-morphogenesis, a similar protocol was used on the coral Favia favus. The tissues roundedup 24 hours after the removal from the adult colony and became explants (formed calluses). Four weeks later complete polyps had developed with a mouth, septae and tentacles.

Oculina patagonica Polyp Culture

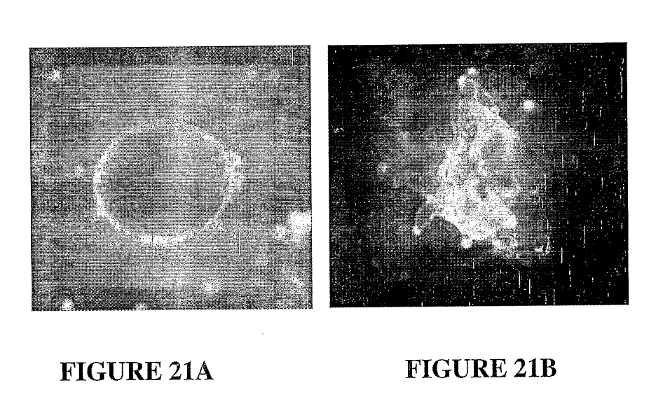

In order to examine the ability of other species to undergo re-morphogenesis, a similar protocol was used on the coral Oculina patagonica. The complete development of the polyp is shown in FIG. 21. The tissues rounded up 24 hours after the removal from the adult colony and became the explants (formed calluses). Two weeks later complete polyps had developed with a mouth, septae and tentacles. By the third week 7/105 fragments developed into polyps.

### Modified Polyp Culture

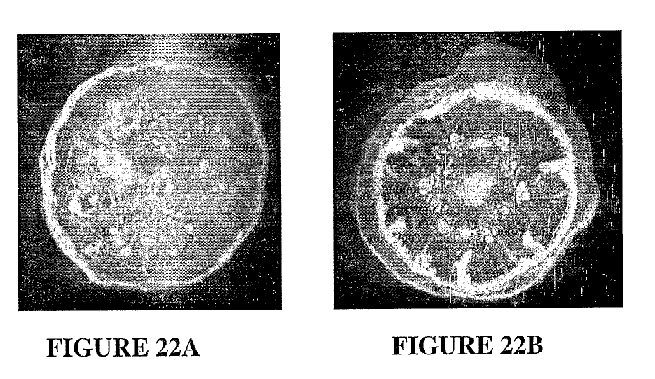

Polyps bleached one week after adding cycloheximide in all the concentrations that were used. In FIG. 22 a healthy coral vs. a bleached coral is shown.

### REFERENCES

Arvedlund, M., J. Craggs, and Pecorelli J. 2003. Coral culture—possible future trends and directions. In Marine ornamental species: collection, culture & conservation, ed. J. C. Cato and C. L. Brown, 233-248. Ames, Iowa: Iowa State Press.
Borneman E. 2000. Coral reef organisms. Issues in Sci. and Technol. 17 (2): 17-18.
Borneman E H. 2000. Aquarium Corals: Husbandry, Selection and Natural History (Foreward by JEN Veron). Microcosm, Ltd. Shelburne. 464 pp.
Borneman E H and Lowrie J. 2001. Advances in captive husbandry and propagation: An easily utilized reef replenishment means from the private sector?
   Bull. of Mar. Sci. 69 (2): 897-913.
Brown, B. E. Coral bleaching: causes and consequences. 1997. Coral Reefs 16: 129-138.
Domart-Coulon I, Tambutté S, Tambutté E and Allemand D. 2004. Short term viability of soft tissue detached from the skeleton of reef-building corals. J. of Exp. Mar. Biol. and Ecol. Vol. 309, 2, 6: 199-217.
Goreau T J, Hayes R L. 1994. Coral bleaching and ocean “hot spots”. Ambio. 23:176-180.
Hyman L H. 1940. The invertebrates: protozoa through Ctenophora.

### New York: McGraw Hill Inc.

Highsmith R C. 1982. Reproduction by fragmentation in corals. Mar. Ecol. Prog. Ser. 7:207-226.
Hoegh-Guldberg 0.1999. Climate change, coral bleaching and the future of the world's coral reefs. Mar. Freshwat. Res. 50, 839-866.
Jokiel P L, Hunter C L, Taguchi S, Watarai L. 1993. Ecological impact of a fresh water “kill” on the reefs of Kaneohe Bay, Oahu, Hawaii. Coral Reefs.
Kaplan, E. L., and P. Meier. 1958. Nonparametric estimation from incomplete observations. Journal of the American Statistical Association 53:457-481.
Kramarsky-Winter E and Loya Y. 1996. Regeneration versus budding in fungiid corals: a trade off. Mar. Ecol. Progr. Ser. 134:179-185.
Krupp D A, Jokiel P L and Chartrand T S. 1992 Asexual Reproduction by the Solitary Scleractinian Coral Fungia scutaria on Dead Parent Coralla in Kaneohe Bay, Oahu, Hawaiian Islands. Proc. of the 7th Int. Coral Reef Symp., Guam, Vol. 1:527-534.
Latypov Y. 2006. Transplantation and cultivation of fragments of coral colonies of various scleractinian species on a reef in Vietnam Russian Journal of Mar. Biol. Vol. 32, No. 6: 375-381(7).
Rinkevich B. 1995. Restoration strategies for coral reefs damaged by recreational activities: the use of sexual and asexual recruits. Restor. Ecol. 3: 241-251
Shafir S, Van Rijn and. Rinkevich B. 2001. Nubbing of coral colonies: a novel approach for the development of inland broodstocks. Aqua. Sci. Conserv. 3, pp. 183-190.
Shafir S, Van Rijn J, Rinkevich B. 2006. Coral nubbins as source material for coral biological research: A prospectus. Aquaculture 259: 444-448.
Stachowicz J J and Hay M E. 1999. Mutualism and Coral Persistence: The Role of Herbivore Resistance to Algal Chemical Defense. Ecol. Vol. 80, No. 6: 2085-2101.
Weeks J M and Svendsen C. 1996. Neutral-red retention by lysosomes from earthworm (Lumbricus rubellus) coelomocytes: a simple biomarker of exposure to soil copper. Environ. Toxicol. Chem. 15, pp. 1801-1805.

1. A method of culturing tissue spheroids from a scleractinian coral species or ecotype, said method comprising the steps of:
a. Excising coral tissue fragments; and
b. Culturing said coral tissue fragments in seawater, at a temperature that is at the low range for said coral species whereby tissue spheroids are formed.

2. A method according to claim 1 wherein said scleractinian coral species or ecotype is obtained from a sea having warm temperature conditions.

3. A method according to claim 2 wherein said scleractinian coral is Fungia granulosa or Favia favus.

4. A method according to claim 2 wherein said coral tissue fragments are maintained at a temperature not higher than about 22° C.

5. A method according to claim 4 wherein said coral tissue fragments are maintained at a temperature of about 19° C. to about 21° C.

6. A method according to claim 1 wherein said scleractinian coral species or ecotype is obtained from a sea having cool temperature conditions.

7. A method according to claim 6 wherein said scleractinian coral is Oculina patagonica.

8. A method according to claim 6 wherein said coral tissue fragments are maintained at a temperature of about 16° C.

9. A method according to claim 1 wherein said scleractinian coral species or ecotype is obtained from a sea having cold temperature conditions.

10. A method of preparing viable polyps from a mature scleractinian coral species or ecotype, said method comprising the steps of:
a. Obtaining coral tissue spheroids according to claim 1; and
b. Incubating said tissue spheroids at an optimal temperature for growth for said coral species or ecotype, whereby mature polyps having mouth, septae and tentacles are obtained.

11. A method of obtaining a second generation of viable polyps in culture, said method comprising the steps of
a. Obtaining mature polyps according to claim 10;
b. Excising coral tissue fragments from said mature polyps; and
c. Inducing re-morphogenesis by incubating said tissue fragments at an optimal temperature for growth for said coral species or ecotype, thereby obtaining a second (F2) generation in culture.

12. A method according to claim 10 wherein said scleractinian coral species or ecotype is obtained from a sea having warm temperature conditions.

13. A method according to claim 12 wherein said scleractinian coral is Fungia granulosa or Favia favus.

14. A method according to claim 12 wherein said coral tissue fragments are maintained at a temperature of about 22° C. to about 30° C.

15. A method according to claim 10 wherein said scleractinian coral species or ecotype is obtained from a sea having cool temperature conditions.

16. A method according to claim 15 wherein said scleractinian coral is Oculina patagonica.

17. A method according to claim 15 wherein said coral tissue fragments are maintained at a temperature of about 16° C. to about 30° C.

18. Non-differentiated soft tissue coral spheroids, capable of being kept viable in culture for at least one month and capable of undergoing re-morphogenesis to coral polyps upon increasing the temperature of the culture.

19. A scleractinian coral line of a single genetic source obtainable by the method of claim 1.

20. Scleractinian coral tissue polyps of a single genetic source obtainable by the method of claim 7.

21. A method for screening the toxicity of a compound comprising:
a. Obtaining coral tissue spheroids, coral polyps or coral lines in accordance with claim 18;
b. Administering said compound to said spheroids or polyps; and
c. Measuring viability or physiological state of said spheroids or polyps;
wherein low viability or compromised physiological state indicate a high toxicity of the screened compound.

22. A method according to claim 21 wherein physiological state is determined by measuring bleaching.

23. A method according to claim 21 wherein viability is determined by neutral red staining.

24. Use of coral tissue spheroids, coral polyps, or coral lines in accordance with claim 18 as model organisms for toxicology screening of compounds.

25. Use in accordance with claim 24 wherein said compounds are selected from the group consisting of drugs, food ingredients, cosmetics and potential environmentally hazardous compounds.
